(19) United States
(12) Patent Application Publication
(10) Pub. No.: US 2015/0064691 A1
(43) Pub. Date: Mar. 5, 2015

(54) METHOD OF INTRACELLULAR INFECTIOUS AGENT DETECTION IN SPERM CELLS

(71) Applicants: Vassilios Tsilivakos, Athens (GR); Aggelos Gritzapis, Athens (GR)

(72) Inventors: Vassilios Tsilivakos, Athens (GR); Aggelos Gritzapis, Athens (GR)

(21) Appl. No.: 14/387,794

(22) PCT Filed: Mar. 29, 2013
(86) PCT No.: PCT/GR2013/000016

§ 371 Date: Sep. 24, 2014

(30) Foreign Application Priority Data
Mar. 29, 2012 (GR) 20120100185

(51) Int. Cl. G01N 33/569

(52) U.S. Cl. CPC G01N 33/56994; G01N 33/56983; G01N 33/56927; G01N 33/56988; G01N 33/56905; G01N 2800/367
USPC 435/5; 435/7.1

(57) ABSTRACT

The present invention describes a method for investigating the presence of chlamydia, viruses and other infectious agents in spermatozoa, with the use of immunofluorescence in combination with flow cytometry. This method is used for the intracellular detection of microorganisms within sperm cells through the use of a DNA easing procedure, as well as for the detection of microorganisms adherent to the surface of spermatozoa.

### TECHNICAL FIELD OF THE INVENTION

The present invention describes a method for subjecting semen, namely sperm-cell populations to analysis in order to detect intracellular viruses, Chlamydia, parasites and other microorganisms inside spermatozoa, in an effort to study and determine the etiology of sub-fertility and of infertility, as well as to prevent congenital infections. The whole procedure is accomplished with use of a special process for the easing of DNA structure, the targeting of microorganisms with antibodies and the evaluation of results with flow cytometry.

### PRIOR ART

Sub-fertility is currently an increasingly frequent problem that affects many families. To address this problem many approaches have been developed, including in vitro fertilization (IVF).

Fallopian tube obstruction, azoospermia and menopause are indisputable causes of subfertility. Oligospermia and premenopause, which are the earlier stages of azoosperimia and menopause are also of interest as they are progressively important factors leading to sub-fertility. Oligospermia in particular, should be a problem easily resolved through IVF, since a single spermatozoon should be enough for conception. In practice however, this is not the case.

For spermatozoa there is a variety of qualitative flaws that have been intensively studied and which particularly affect two evaluation parameters of sperm quality: morphology and motility. Motility provides embryologists in IVF attempts with only approximate information for the suitability of spermatozoa for micro-fertilization. Sperm morphology on the other hand, is a much more accurate criterion for the evaluation of sperm as suitable or not for fertilization. However, sperm morphology cannot be used directly as a measure for the selection of good spermatozoa to be used in IVF as the spermatozoa used for morphological characterization of a sperm sample are destroyed in the process. According to the above, clearly there are many ways to evaluate sperm samples (World Health Organization reference values for human semen characteristics, Human Reproduction Update 2009) which are taken into account upon observing subfertile couples, many of whom will proceed to use IVF methods.

The final characterization of sperm samples will be the result of a combined evaluation of all the specific morphological abnormalities detected on each examined spermatozoon. Spermatozoa characterized by the absence of these abnormalities are classified as “suitable” or “normal”. Furthermore, physiological values have been also given through teratozoospermia index (TZI) which measures the average value of morphological abnormalities per abnormal spermatozoon. It is also possible to assess sperm quality by measuring of the percentage of apoptotic spermatozoa in the sample, that is spermatozoa that have entered the process of programmed cell death.

However, every sperm abnormality can be attributed to a specific cause. Such causes could be microbes, for example in cases of chronic prostatitis, or other factors such as smoking, obesity, excessive physical exercise, high temperatures etc. On the contrary, viral factors as a cause of male subfertility have not greatly concerned the medical community up until now. In 2004 and 2005, in two American Journal of Reproductive Immunology publications, researchers in Locus Medicus S.A. (including V. Tsilivakos, inventor of the present invention) described a correlation between high numbers of Natural Killer (hereinafter referred to as NK) lymphocytes in the blood of women with a history of subfertility and/or miscarriges and the presence of subclinical herpes viremia (HSV1-2, EBV, CMV, HHV6 and HHV7).

Following that, the inventors observed in aborted material that NK lypmhocytes mostly congregated at the implantation site, while the blood NK levels of these women were normal. According to the theory of the inventors, this can be explained if the embryos in these cases were by themselves antigenic due to the presence of viral (at least herpetic) antigens, originating from the male through the sperm cells including spermatozoa. These antigens would be expressed and presented to the woman's immune system by fetal cells causing the NK reponse.

However, when we attempted to establish the presence of viral elements through classical diagnostic methods such as sperm morphology or other parameters of semen analysis we found that it was not feasible.

### Problem to be Solved

When a couple reaches an infertility clinic, the correct approach is to investigate the cause of the problem. Many symptoms and signs have been implicated as factors involved in infertility, but many of them are not primary causes, they are themselves the result of other factors, usually infectious in nature, the eradication or the containment of which may contribute to treatment. For example, we know that the presence of chlamydia or other microorganisms in the fallopian tubes can lead to tubal obstruction, temporary at first, but which can be permanent if therapeutic intervention does not take place. Similarly, DNA fragmentation of sperm cells including spermatozoa due to cell apoptosis, is often the result of bacterial or possibly of viral infection of various parts of the male genital tract as well as of other factors such as of oxidative stress.

Success in tackling the problem of couple infertility depends on the absolute accuracy of the explanation of the primary causes of the problem, whether the couple achieves natural conception or uses some sort of assisted reproduction. Unfortunately, only recently was the clinical significance of viral presence in semen acknowledged by individual researchers (but not by the medical community as a whole), a fact that becomes even more clinically relevant, if indeed the viruses are contained within spermatozoa that will eventually fertilize the ovule. In this case, the viral infection could pass from spermatozoon to zygote through vertical transmission resulting in the simultaneous proliferation of fetal and viral cells which could then populate organs and tissues where they are known to cause birth defects, such as is the case of herpes virus in the nervous system or other viruses in the cardiovascular system. In our opinion, in order for the adverse effects of the presence of an infectious (viral) factor to take place, there is requires a failure of the immune system to fight the infection. By contrast, if the woman's defense mechanism is able to confront the infectious (viral) factor through recognition of viral antigens, this would lead to the destruction of the virus-infected fetal cells.

Mechanisms of embryonic cell rejection may involve those that take place via NK cells, for which so much fuss has been made over the past 25 years, concerning their role in the process of first trimester miscarriages of immunological etiology. This could be true at least in the case of herpes viruses, against which the activation of NK cells is a subsequent reaction. Furthermore, in a recent publication, the inventors described a high incidence of infertility among Greek teachers. This could be attributed to their high exposure to childhood viral infections i.e. higher concentrations of viruses. In addition, we observed a higher occurrence of miscarriages in couples that are in long-term relationships, which could indicate a stronger immune memory on the part of the woman. That would lead to a stronger immunologic response of the female immune system towards the “well-known” viruses of the male partner, resulting in the rapid destruction of infected embryonic cells. Unfortunately, there have not yet been any studies internationally, concerning embryonic antigenicity. However, the inventors believe that the developing embryo expresses few or none of the male only antigens, which are the antigens developed by the male and which antigens the female organism would not create so as to recognize them as self—at least until the end of the first trimester.

Regarding HLA molecules that differ between spouses, we know that their expression is downregulated in fetal cells that come into direct contact with the immune system of the woman. So the question is, which foreign antigens does the female organism attack during a first trimester miscarriage or during a miscarriage that occurs much earlier and cannot thus be perceived through a delay of the menstruation. In the future, it is necessary that the international scientific community addresses the degree of clinical significance as well as the appropriate treatment of each case of subclinical viral infection of the male (be it sporadic or chronic) which could result in vertical transmission of viral antigens to the fetus.

The inclusion of this factor by the inventors of the present invention has created the opinion by the inventors that the failure to consider non-viruses and other contaminants in sperm cells including spermatozoa, corresponds to an improper and inadequate study of infertility.

Therefore, there is a great need for a method for the detection of infectious agents in sperm cells including spermatozoa with high sensitivity and specificity, but unfortunately, until now, this has not been technically possible.

### Other Approaches

Until now, methods for detecting microorganisms in semen such as chlamydia, have used immunofluorescence (slide mounted cell suspension), but this is a method of very low sensitivity. The serological ELISA method which detects circulating antibodies in the blood is also frequently used, but lacks any information on the localization of the infection.

Microorganisms including mycoplasmas are presently detected by semen cultures. However, in order to culture intracellular infectious agents, the use of specialized cell lines and cell culture equipment as well as strict safety laboratory regulations are required. These increased requirements make it almost impossible to apply this technique on a daily basis.

In recent years the use of molecular techniques (mainly Polymerase Chain Reaction or PCR) can be used for the detection of chlamydia and infectious agents following DNA extraction from sperm or washed cellular components of semen. But the detection of intracellular pathogens in the internal of sperm cells including spermatozoa via scanning electron microscopy has not been described.

Finally, electronic microscopy can detect the presence of microbes or viruses attached solely to the outer surface of the cell membrane but not of intracellular ones.

For the detection of viruses in semen, the most effective approach available today which is characterized by high sensitivity and specificity is PCR, of washed cellular components of semen. The main drawbacks of this technique, however, is that it cannot discriminate between extra- or intra-cellular parasites, i.e. whether the microorganisms detected are located on the outside or the inside of spermatozoa and also it cannot indicate the specific cell-type infected, whether it is spermatozoa or other cell types contained in semen, such as for example leukocytes or sperm cell progenitors.

Additionally, serological detection of antibodies against viruses and/or toxoplasma does not provide any information concerning the localization of the infectious agent on the inside or on the outside of sperm cells or on any other cellular component of semen.

Finally, detection of microorganisms within the cells is possible through fluorescent or chromogenic in situ hybridization. However, this method is of lower sensitivity, it is more time consuming and more expensive than that described in the present invention.

The method disclosed and described in the present invention, for the first time allows the intracellular detection of infectious agents, that is, those located within (on the inside) of sperm cells including spermatozoa.

The patent application of Stuart et al., US pat. Application pub. No. US2006/0099661 A1 entitled “Detection and quantification of intracellular pathogens” focuses on the detection of chlamydia on both the surface of the cell as well as intracellularly, mostly in peripheral blood cells but also on cells of other biological fluids including sperm. In this patent the inventors describe the following three basic steps:

a) obtaining the biological fluid
b) use of a primary antibody for specific recognition of a chlamydial antigen on the cell surface or intracellularly and
c) analysis of the sample using flow cytometry,

and thus, they describe that by the use of the proposed methods described in the patent they succeed to detect of chlamydia in peripheral blood cells.

In addition, according to the described method of chlamydia detection within lymphocytes by use of the chemical TRITON-X, the authors of US pat. Application pub. No. US200610099661 A1 entitled “Detection and quantification of intracellular pathogens” claim that the method could be extended to the detection of chlamydia also within other cells, including sperm cells.

In order to test that assumption made in the said prior art docment, the inventors of the present invention performed the experiments described in US2006/0099661 A1, according to the experimental method proposed by the inventors, Elizabeth S. Stuart and Lloyd H. Semprevivo. However, despite the assertion expressed in US2006/0099661 A1 concerning the extension of the method to the detection of chlamydia within sperm cells, as we show in the histograms that follow as FIG. 1, the inventors found that chlamydia detection intracellularly in sperm cells is impossible following the experimental technique described by Stuart and Semprevivo and this on a known sample that was characterized as “very positive” for the presence of chlamydia by the method disclosed by the inventors in the present invention.

According to the results of the inventors, the curve representing the sperm sample used for detection of chlamydia according to the procedure described by Stuart and Semprevivo did not show a shift to the right compared to the control (FIG. 1. B)—the two curves are indistinguishable. This means that the method did not detect the presence of chlamydia in the sample. The inventors of the present invention attribute the failure of the method by Stuart and Semprevivo to detect chlamydia within sperm cells to the absence of enzymatic treatment of cells (with DNAase), which treatment the inventors of the current invention suggest and have proven to be an essential step of the procedure for the detection of any infectious factor (bacterial or viral) inside sperm cells and which procedure is central to and an essential element of the method of the present invention.

Furthermore, the results of the inventors of the present invention showed that the method of Stuart & Semprevivo is characterized by low sensitivity and low specificity as far as the study of sperm is concerned, especially when a direct fluorescence method is used. This is illustrated in FIG. 2, which shows that by using the protocol proposed in the patent application of Stuart & Semprevivo, an anti-CD3 antibody with specificity for mouse antigen is detected inside the negative control sperm sample while theoretically this antibody should not bind to any antigen in the sperm cells (low specificity). In other words, the proposed method of Stuart & Semprevivo produces erroneous and misleading results.

The above results prove that the method proposed by the patent application US 2006/0099661 A1 of Stuart et al. for detecting chlamydia intracellularly in sperm cells does not provide a solution to the problem noted by the present invention.

### DISCLOSURE OF THE METHOD OF THE PRESENT INVENTION

According to the inventors' view, a circumstantial study of putative infectious factors in sperm is imperative, whenever there is a clinical history of one or more of the following: early pregnancy failure, biochemical pregnancy, oligospermia, asthenospermia or teratospermia, unsuccessful IVF attempt, sub-fertility, or for sub-fertility prevention in general.

Especially for sub-fertility prevention, a primary focus is Chlamydia detection in spermatozoa and we choose to do so in combination with spermiogram and semen cultures with a view to detecting the existence of other microbes also.

The current invention describes a method of detection and study of the presence of viruses, chlamydia, parasites and other microorganisms inside the spermatozoa, by using direct or indirect immunofluoresence methods, followed by visualization and evaluation with flow cytometry.

The current invention describes that this detection is made within the cells, for detecting microorganisms that lie inside the spermatozoa. The method suggested in the current invention is the immunofluoresence combined with assessment of the result with flow cytometry, with use of a special treatment of easing DNA, for example DNA digestion.

It is characteristic of the current invention that the method described for the investigation of the presence of viruses, Chlamydia, parasites and other infectious pathogens intracellularly in spermatozoa comprises the following steps:
Easing of the dense structure of DNA of the sperm cells including spermatozoa),
direct or indirect intra-sperm cell immunofluorescence,
visualization and evaluation of the result with flow cytometry.

We would like to stress that it is critical that DNA easing of DNA dense structure takes place before immunofluorescence, for the rendering of target antigens detectable by specific antibodies.

It is advantageous that the step of visualization and evaluation of results by flow cyttometry comprises the incubation of cell pellets with 7-aminoactinomycin D (7AAD) in WB in order to enable the discrimination between 1N and 2N cells.

Preferably, the method described in the present invention is carried out for the determination of the causes of sub-fertility, of early pregnancy failure or miscarriage or fetal loss. In addition, it can be used for the prevention and studying of congenital infection or for the prevention of vertical transmission and for the detection of inflammation and infections of the male genital system, for example epidydimitis.

The method of the present invention can also detect the specific existence of one of the following pathogens: Cytomegalovirus (CMV), Herpes Simplex Virus I (HSV I), Herpes Simplex Virus II (HSV II), Epstein Bar Virus (EBV), HHV6, HHV7, HHV8, Parvovirus 19, Hepatitis B Virus (HBV), Hepatitis C Virus (HCV), Coxsackie Virus, Human Immunodeficiency Viruses (HIV-1, HIV-2), Adeno-associated Virus (AAV), Rubella Virus, HPV, Chlamydia, Toxoplasma and Norovirus.

In particular, regarding the detection of Chlamydia in spermatozoa, the current method could be combined with a spermiogram and semen cultures for the detection of other pathogens, with the significant advantage that these may be carried out on the same sample or on different samples.

Preferably, the easing of the DNA structure of spermatozoa′ DNA which is very dense is performed with DNA digestion and results in DNA fragmentation.

It is advantageous that DNA digestion is accomplished with an enzyme which breaks DNA.

For example, this enzyme could be DNase I.

For immunofluoresence, any suitable fluorescent antibody or anti antibodies can be used. For this purpose, any fluorochrome can be used including any one of the following that are known today: Fluorecein-5-isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5), peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, or Alexa Fluor 700.

The method of the present invention may include an additional step for detecting surface antigens.

The invention describes also the development and use of a kit for the intra-spermatozoan detection of chlamydia, viruses, parasites and other pathogens inside spermatozoa using the method of the present invention. The kit should necessarily comprise a substance that can ease DNA of spermatozoan cells. This substance for example could be an enzyme that digests DNA; for example the enzyme could be DNase I. In addition the kit disclosed in the invention should comprise one or more antibodies against the specific pathogens whose presence is requested to identify. The above antibodies can be directly labeled with a fluorochrome such as these described above. If the specific antibodies for the pathogens are not labeled, a second fluorochrome labeled or biotinylated antibody which recognizes the first, should be included.

### DESCRIPTION OF THE FIGURES

The current invention can be illustrated by use of the following Figures.

In FIG. 3, the intra-spermatozoan detection with flow-cytometry of C. trachomatis and HSV antigens is illustrated. In FIGS. 3A, 3B, 3C the detection of specific antigens is observed after DNase I digestion. In contrast in FIGS. 3D, 3F, 3G where DNase I digestion has not preceded, there is no such detection.

In FIG. 1 the failure of Chlamydia detection inside spermatozoa according to the protocol described in patent apl. number US 2006/0099661 A1 by Stuart et al. (for other cellular types), is shown. The same sample is characterized as strongly positive after DNA digestion according to the current invention (data not shown).

FIG. 2 illustrates the loss of antibody specificity, according to the protocol described in patent apl. number US 2006/0099661 A1 of Stuart et al. (for other cellular types). An antibody which is against mouse CD3 binds, non specifically binds to spermatozoa, causing the fluorescence shift to the right.

### EXAMPLE

An example of an embodiment of the present invention follows:

1. Fixation of Sperm Cells Including Spermatozoa

Following collection and liquidation of semen, spermatozoa are spun down and fixed with 4% Paraformaldehyde (PFA) at 4° C. for 30 min. PFA works by cross linking proteins, thereby inactivating pathogens and immobilizing putative auto-antibodies that are already linked to spermatozoa, thereby rendering them detectable, if this is desired. In some rare cases, some antigenic epitopes may be altered or destroyed making them undetectable by certain antibodies. In such instances, surface staining may be performed prior to fixation. In addition, PFA fixation preserves the physical characteristics of cells: i.e. following fixation the cells exhibit the same scatter characteristics shown, during analysis done with flow cytometry. In addition PFA fixation allows the subsequent application of either an extracellular or intracellular staining procedure.

2. Important Notice:

Before incubating a particular antibody with the pathogen it will recognize and bind with, it is of vital importance to the procedure and it is necessary to carry out first a step to ease, or loosen, the spermatozoa′ dense DNA structure. This process can be achieved by any method which may loosen DNA structure, among others with DNA digestion which has the ability to loosen the dense structure of spermatozoa′ DNA. This process of loosening DNA may be done with any other means that may achieve the same result, either mechanic, thermic, electrolytic method or withuse of reducing agents such as β mercaptoethanol, Dithiothreitol, tris(2-carboxyethyl)phosphine.) Such may be the use of an enzyme for DNA digestion. In the specific example presented in the present specification, an example of such a substance is a DNA digestion enzyme. In the specific example used in the present specification, as such DNA digestion substance we have used the enzyme DNAse I. FIG. 3 illustrates the failure of pathogen detection without the step of DNase I digestion.

3. Intrasperm Staining (Indirect)

Indirect staining is used in our example because it is less costly than direct staining. However, the present invention can also function if it alternatively utilizes directly conjugated antibodies as described below.

A. A fraction of cells is spun down, resuspended, and incubated for 30 min with 100-500 μl Phosphate Buffer Saline (PBS) containing 4% PFA and 0.1% saponin (medium A). The cells are then washed with 2 ml PBS containing 0.1% saponin and 2% Fetal calf serum (FCS) (wash buffer-WB). The supernatant is discarded and the pellet incubated with 100-500 μl PBS containing 10% Dimethyl sulfoxide (DMSO) and 0.1% saponin for 10 min. Following a wash with WB, the pellet is fixed with 100-500 μl of medium A at 4° C. Following a 10 min incubation, the cells are washed with WB, the supernatant is discarded and the pellet is resuspended, and incubated for 30 min with DNase I (500 μg/ml) at 37° C. Finally, the cells are washed with WB, the supernatant is discarded and the pellet incubated with titered amounts of the particular antibody specific for one of the following pathogens:

a. Cytomegalovirus (CMV)
b. Herpes Simplex Virus I (HSV I) and/or Herpes Simplex Virus II (HSV II)
c. Epstein Bar virus (EBV)
d. HHV6
e. HHV7
f. HHV8
g. Parvovirus 19
h. Hepatitis B virus
i. Hepatitis C virus
j. Coxsackie virus
k. HIV (HIV I, HIV II)
l. Adeno associated virus (MV)
m. Rubella virus
n. HPV
o. Norovirus
p. Chlamydia
q. Toxoplasma.

The incubation of cells with antibodies, takes place either in separate tubes for each one of the pathogens or in the same tube, which allows the simultaneous detection of pathogens, provided that there are directly conjugated antibodies with discrete fluorophores, that emit colors which contrast to each other.

After 30 min incubation at 4° C., the cells are washed with WB and the supernatant is discarded.

B. The cell pellet is resuspended and a new incubation with 50 μl of a polyclonal fluorophore conjugated antibody against immunoglobulins from the animal from which the first antibody was developed, follows. Any fluorophore can be used. The following are the most known nowadays fluorophores, which are mentioned indicatively and which should not considered to restrict our choice of fluorophore:

Fluorecein-5-isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5), peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Alexa Fluor 700.

The incubation of the cells for 30 min at 4° C. follows and then the cells are washed with 2 ml WB. If it is necessary to perform a leukocyte study, the procedure goes on to next step. Alternatively, the procedure goes to the step where the cells are harvested.

### Optional Step of Leukocyte Staining

If we face the question whether there are microorganisms present in the leykocytes of the semen, the samples will be incubated with a directly-conjugated antibody against a leukocyte antigen to assess the possible presence of pathogens within the leukocytes. The fluorophore attached to this antibody must differ from the other fluorophores used for pathogen detection. A 30 min incubation at 4° C. follows and then follows another 2 ml WB wash. The supernatant is discarded, and the cells are resuspended. Furthermore, the discrimination between 1N and 2N cells is feasible after incubation of cell pellets with 7-aminoactinomycin D (7AAD) in WB. After 5 min incubation, the cells are ready for acquisition in a Flow Cytometer.

### Direct Immunophenotyping of Pathogens

Alternatively, if the specific antibodies against putative pathogens are directly conjugated with fluorophores, stage B, as described above, can be omitted. In addition, each specific antibody can be conjugated with biotin and their detection achieved by subsequent incubation with a streptavidin-fluorophore complex. As an alternative to biotin, any other mode of fluorophore conjugation can be used. Any fluorophore can be used including, but not limited to the following known fluorophores: Fluorecein-5-isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5), peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Alexa Fluor 700.

4. Acquisition and Evaluation of the Results

The samples are acquired in a flow cytometry apparatus and the data analysis is performed using suitable software. The cells are gated using region combinations based on their size and complexity and/or the expression of antigens (such as leukocyte antigens). The analysis focuses on the putative presence of pathogens within the spermatozoa or within other cellular components of sperm. Moreover, the detection of such pathogens in the leukocytes is feasible using suitable regions based on the expression of leukocyte antigens.

B. Surface Immunostaining

In addition to intra-spermatozoan staining, the detection of putative extracellular pathogens is also feasible. A second fraction of cells is subjected to centrifugation, the supernatant is discarded and the cells are equally distributed to tubes which contain tittered amount of a particular antibody, specific for each pathogen. Following a 30 min incubation at 4° C., the tubes are washed with PBS containing 2% FCS (PBS-2% FCS), and the supernatant is discarded. Next, the cells are re-suspended and incubated once more in 50 μl of a polyclonal fluorophore-conjugated antibody against immunoglobulins of the animal from which the first antibody was developed. Following a 30 min incubation at 4° C., the tubes are washed with PBS containing 2% FCS (PBS—2% FCS), and the supernatant is discarded. The cells are re-suspended and placed in a flow cytometry apparatus for acquisition and analysis.

### DETAILED DESCRIPTION OF FIGURES

In particular:

In the 2D scatter plot of FIG. 3, the size of spermatozoa (FSC-H) is presented with respect to their complexity (SSC-H), where an enclosure region R is defined, so that a spermatozoa-enriched cell population can be studied.

The specific fluorescence for the antibodies that detect one chlamydia antigen and one herpes antigen respectively, according to the above procedure without DNAse use, is presented in histograms 3E and 3F in FIG. 1. Additionally, in the same histograms, the fluorescence of the control by the labeled anti-antibody is also shown. Comparison of these two curves shows that there is no antigen detection, i.e. no detection of the infectious agent.

The same parameters from the study of spermatozoa in the same enclosure region R, are illustrated in histograms 3A, 3B and 3C of FIG. 3, but in this case the DNAase incubation has been comprised as detailed above. The data analysis shows that, in the presence of DNAse, the detection of infectious agents in spermatozoa is feasible, as manifested by the fluorescence shift to the right, as compared to the control (which is processed by the same procedure omitting the use of infectious agent-specific antibody).

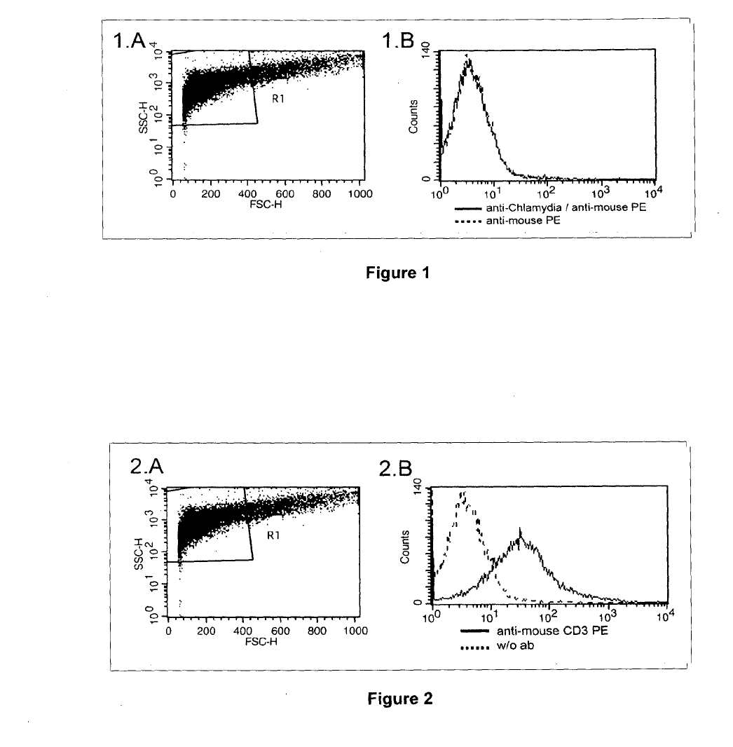

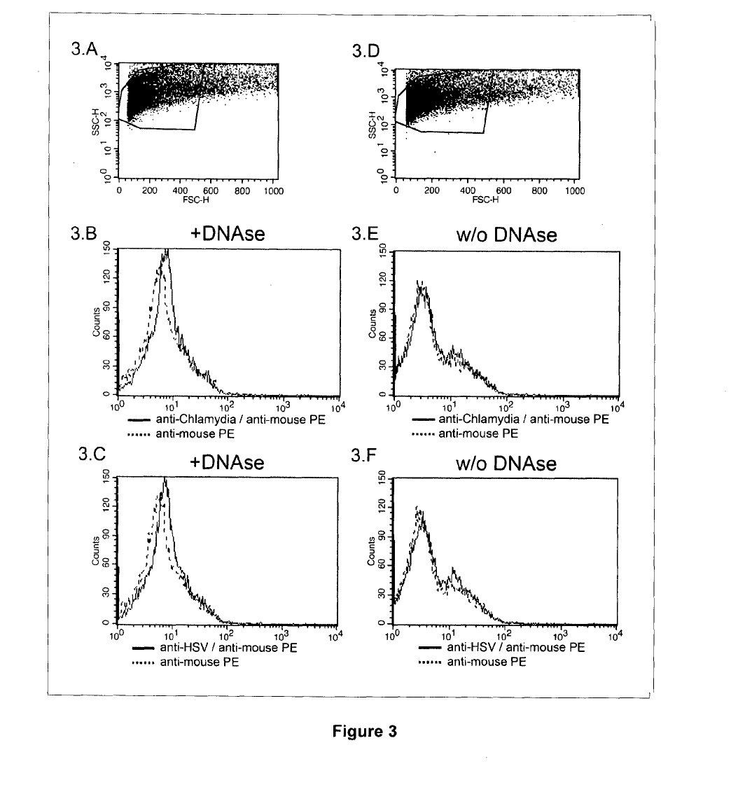

FIGS. 1 and 2 are described in detail above, in the prior art chapter of the present specification.

### Advantages of the Method of the Present Invention

The main advantage of the method for the detection of infectious agents inside sperm cells including spermatozoa as described in the present invention, is its high sensitivity, provided that the technique and conditions for cell fixation, membrane permeabilization and, most importantly, the enzymatic loosening of the dense structure of DNA with a DNA digestion enzyme are met. In a parallel investigation made by the inventors of the present invention, it was found that this method detects the presence of an infectious agent even in cases where a molecular (PCR) detection test on the same sample is negative. Furthermore, with the use of a technique which uses special beads and can match fluorescence levels to the number of antigens, it is possible to construct a curve that correlates fluorescence intensity with the number of microorganisms or viruses per infected sperm cells including spermatozoa. The method proposed by the invention allows the study of large numbers of cells (eg 20,000) per sample and the retrospective retesting of samples. This quantitative superiority as provided by the proposed method minimizes the likelihood of a false negative result (failure of detection of the infectious agent).
The high specificity of the method has been established and is ensured through the use of negative controls and, of course, through the use of monoclonal antibodies (reagents that specifically detect and react with a single specific site of a particular molecule for each microorganism or virus). In addition, through scatter characteristics evaluation and/or with the help of supplementary antibodies, (i.e. anti-CD45), the presence of microorganisms in sperm cells including spermatozoa can be confirmed. It is also clearly shown that the infectious agent is attached to the outside surface of the cell membrane and not to the inside of the sperm cells including spermatozoa; such information can be of great clinical significance.
The described method allows assessment of the efficacy of an antibiotic or antiviral treatment. It is useful to monitor pathogen status by determining the regression of infection as indicated by the diminishing numbers of detected microorganisms in the sample (eg chlamydia after tetracycline treatment).
Fixed samples can be safely stored for long periods of time until tested. The shipment of fixed samples is also feasible. Since the method described in the present invention for the study of semen requires the use of flow cytometry, it is not possible for laboratories lacking this technology to utilize it. To deal with this issue, the method described in the present invention allows for the safe transfer or shipment of fixed samples (between different locations and laboratories) without loss of sensitivity or specificity. In addition, the ability of safe sample transportation allows retesting of a sample in a different laboratory in the event of a technical failure of the flow cytometer. Finally, the resulting data are stored electronically by the device and are therefore available for reassessment at any time.
The described method is characterized by a very low cost which is certainly lower than that of the equivalent PCR tests.
The described method is also characterized by a very fast laboratory turnaround time as the test results are available on the same day.

Also, the present invention describes, for the first time, a method for detecting intracellular infectious agents in sperm cells including spermatozoa using a specific immunofluorescence technique and evaluating test results by flow cytometry.

The intracellular analysis of spermatozoa becomes possible by use of a DNA digestion enzyme that “loosens” DNA structure inside the cells. The inventors of the present invention have attributed the inability of the reagents (antibodies) to detect microorganisms (target antigens) within the spermatozoan head up until now, to the particular and very high concentration of DNA that is present in that region of the cell. As a result, the inventors of the present invention deem it necessary to “loosen” the DNA through digestion in order to clear a path for antibodies to come into contact and bind to target antigens (microbes).

As far as the detection of microorganisms intracellularly or on the outside surface of the cell membrane is concerned, the inventors of the present invention believe that these are two different kinds of approach, with different clinical interpretations. For example, the inventors attribute subclinical viremias and chlamydiaemias to progenitor sperm cell infection through penetration of the blood-testis barrier. On the other hand, the membrane localization of microorganisms is mainly associated with infection of the sperm release pathway (epididymis, prostate, urethra).

Regarding natural conception, the zygotic cell does not appear to be protected against vertical transmission of intracellular infectious agents, that is, direct transmition of infectious agents by the spermatozoon to the fetus, while the transmission of membrane bound infectious agents can be more easily deterred. For example, according to Aynaud et al. (Frequency of herpes simplex virus, cytomegalovirus and human papillomavirus DNA in semen. Aynaud O et al. Int J STD AIDS. 2002 August; 13 (8):547-50), seminal plasma prevents viral attachment to the cell membrane.

The inventors of the present invention consider the infectious agents on the cell surface to be a relatively minor risk factor for vertical transmission, due to the effects of such factors as seminal plasma, antibodies, proteases, etc. In contrast, they consider vertical transmission of intact intracellular infectious agents to the fetus to be of great risk for the development of problems such as congenital diseases, infertility or early miscarriage.

Especially during natural conception (not intracytoplasmic sperm injection), only the head of the spermatozoon enters the ovum while the rest of the cell (which accounts for most of the spermatozoon cell surface) is excluded. As a result, intracellular infectious agents unavoidably enter the ovum while, on the other hand, the same is not true for membrane-bound microorganisms, as the spermatozoon cell membrane remains outside the zygote during fertilization.

As a result, the inventors of the present invention consider the investigation of intracellular infectious agents in the study of vertical transmission from spermatozoon to fetus of the outmost importance.

In Table 1 below we present data from samples tested in the laboratories of LOCUS MEDICUS S.A. as well as preliminary results from the laboratory of cell biology and immunology of LOCUS MEDICUS S.A. from a five-month period.

| | Total No. of | POSITIVE | | VERY POSITIVE | |
|---|---|---|---|---|---|
| | Samples | (N) | (%) | (N) | (%) |
| sCT | 310 | 204 | 65.81 | 14 | 6.86 |
| cCT | 329 | 219 | 66.57 | 49 | 22.37 |
| CMV | 273 | 125 | 45.79 | 19 | 15.20 |
| EBV | 243 | 59 | 24.28 | 1 | 1.69 |
| HSV I/II | 242 | 103 | 42.56 | 7 | 6.80 |

Table 1 shows Infectious agent detection on sperm samples using flow cytometry. sCT: Membrane-bound C. trachomatis, cCT: Intracellular C. trachomatis, CMV: Cytomegalovirus, EBV: Epstein Barr virus, HSV I/II: Herpes simplex virus.

We consider samples in which positive spermatozoa are detected in more than 5% of the sample as “very positive”.

The above results show that a significant percentage of samples tested was found to be infected by intracellular chlamydia and/or viruses. The detected intracellular infection could not otherwise be found. A positive result (i.e. detection of infection) enables the infection to be treated immediately with the appropriate antibiotics, as shown in Table 2 below.

TABLE 2
Number of cases showing significant reduction of chlamydial load
before and after antibiotic treatment for C. trachomatis infection
in “very positive” samples with flow cytometry. sCT: Membrane-bound
C. trachomatis, cCT: Intracellular C. trachomatis

| | | Significant reduction of chlamydial load | |
|---|---|---|---|
| | Total no. of Cases | (no. of cases) | % |
| sCT | 37 | 20 | 54.05 |
| cCT | 37 | 27 | 72.97 |

More specifically, Table 2 shows that in 20 out of a total of 37 (54.05%) “very positive” samples where membrane-bound C. trachomatis was detected, the chlamydial load was reduced following antibiotic treatment. Furthermore, Table 2 shows that in cases of intracellular detection of C. trachomatis, the percentage of samples which showed a reduction of chlamydial load was even higher, as 27 out of 37 (72.97%) cases showed improvement after treatment with antibiotics.

1. Method for the investigation of the presence of viruses, chlamydia, parasites and other infectious pathogens intracellularly in spermatozoa, which method comprises the following steps:
A procedure for the
easing of the dense structure of the DNA of the sperm cells including spermatozoa,
direct or indirect intra-sperm cell immunofluorescence,
visualization and evaluation of results by flow cytometry,
and which method is characterized by the fact that the step of easing of the dense structure of the DNA must necessarily take place before immunofluorescence.

2. Method according to claim 1 wherein the step of visualization and evaluation of results by flow cyttometry comprises incubation of cell pellets with 7-aminoactinomycin D (7AAD) in WB in order to enable the discrimination between 1N and 2N cells.

3. Method according to any one of claim 1 or 2, which is used for the determination of the causes of infertility, of early pregnancy failure miscarriage or fetal loss and for the prevention of congenital infections or for the prevention of vertical transmission and also for the investigation of inflammation and infections of the male genital system.

4. Method according to any one of claims 1 to 3, which is used specifically for the investigation of one of the following infectious agents: Cytomegalovirus (CMV), Herpes Simplex Virus I (HSV I), Herpes Simplex Virus II (HSV II), Epstein Bar Virus (EBV), HHV6, HHV7, HHV8, Parvovirus 19, Hepatitis B Virus (HBV), Hepatitis C Virus (HCV), Coxsackie Virus, Human Immunodeficiency Viruses (HIV-1, HIV-2), Adeno-associated Virus (AAV), Rubella Virus, HPV, Chlamydia, Toxoplasma and Norovirus.

5. Method according any one of claims 1 to 4, which is used for the investigation for the presence of chlamydia in spermatozoa in combination with spermiogram and cultures using the same sample or on different samples.

6. Method according to any one of claims 1 to 5, where the easing of the very dense DNA structure of spermatozoa DNA is performed with DNA digestion.

7. Method according to claim 6, where DNA digestion is accomplished with an enzyme that breaks DNA.

8. Method according to any one of claim 6 or 7, where the enzyme that breaks DNA is DNase I.

9. Method according to any one of claims 1 to 8, where any appropriately labeled anti-antibody can be used for fluorescence.

10. Method according to claim 9, where for fluorescence any fluorochrome can be used, among which any one of the following: Fluorecein-5-isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5), peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Alexa Fluor 700.

11. Method according to any one of claims 1 to 10, where an extra step of external immunofluorescense is included.

12. Kit for the investigation of the presence of viruses, chlamydia, parasites and other pathogens inside spermatozoa, which comprises at least the following:
a substance that can ease DNA of spermatozoan cells,
one or more antibodies appropriate for the use against the infectious agents of interest.

13. Kit according to claim 12, where the DNA digestion substance is any DNA digestion enzyme.
